(19) United States
(12) Patent Application Publication
(10) Pub. No.: US 2026/0080993 A1
(43) Pub. Date: Mar. 19, 2026

(54) METHODS, DEVICES, AND MEDIUMS FOR GENERATING REPORTS FROM CLINICAL DATA

(71) Applicant: IWE TECHNOLOGY (BEIJING) CO., LTD., Beijing (CN)

(72) Inventor: Mengjie GUO, Beijing (CN)

(73) Assignee: IWE TECHNOLOGY (BEIJING) CO., LTD., Beijing (CN)

(21) Appl. No.: 19/398,025

(22) Filed: Nov. 23, 2025

Related U.S. Application Data
(63) Continuation-in-part of application No. PCT/CN2025/082041, filed on Mar. 12, 2025.

(30) Foreign Application Priority Data
Apr. 8, 2024 (CN) 202410411133.1

(51) Int. Cl. G16H 15/00; G16H 10/20

(52) U.S. Cl. CPC G16H 15/00; G16H 10/20

(57) ABSTRACT

The present disclosure relates to a method for generating a report from clinical data. The method includes: via a reporting generation platform, constructing a reporting generation project corresponding to clinical data to be processed and assigning corresponding roles and permissions to processing personnel; constructing, by the statistical analysts, a template pending review based on a clinical trial design and publishing the template pending review. The method includes: performing, by the reviewers, online review on the templates pending review and add online collaborative annotation, enabling the statistical analysts to obtain a template pending application by updating the template pending review based on the online collaborative annotation; performing, by the annotation specialists, template annotation on the template pending application, enabling the reporting generation platform to automatically generate a corresponding SAS program based on the template annotation; and performing the SAS program to obtain a report pending application.

### CROSS-REFERENCE TO RELATED APPLICATIONS

This application is a Continuation-in-part of International application No. PCT/CN2025/082041, filed on Mar. 12, 2025, which claims priority to Chinese application No. 202410411133.1, filed on Apr. 8, 2024, the entire contents of which are incorporated herein by reference.

### TECHNICAL FIELD

The present disclosure relates to the field of pharmaceutical research and development data analysis technology, and in particular to, a method, a device, and a storage medium for generating a report from clinical data.

### BACKGROUND

Pharmaceutical research and development data analysis is a complex, interdisciplinary field spanning medicine, statistics, computer science, and other related disciplines. This field has developed domestically for over two decades, with the industry scale consistently in a growth phase. However, there are very few specialized programs in domestic universities, creating a high demand for interdisciplinary talents. Moreover, as the vast majority of tasks remain heavily reliant on manual effort, a significant talent shortage persists to this day. The pharmaceutical research and development data analysis imposes exceptionally high-quality standards and demands an extremely low error tolerance. Furthermore, industry practice typically requires that all computational results undergo an “independent dual-programming” quality control process. This mandates that two analysts independently develop and execute programs, and their results must achieve 100% concordance for approval. Consequently, there is a very high dependency on personnel, and a significant amount of working time is required for program execution and quality control.

Regarding clinical data submission, with the advent of the electronic submission era, the U.S. Food and Drug Administration (FDA) mandated compliance with the Electronic Common Technical Document (eCTD) format in 2016, following analogous requirements of the European Union imposed on pharmaceutical manufacturers in 2014. Through nearly two decades of development and enhancement, the Clinical Data Interchange Standards Consortium (CDISC) has developed a plurality of modules to support clinical data standards: the Standard for the Exchange of Nonclinical Data (SEND), the Study Data Tabulation Model (SD™), the Analysis Data Model (ADaM), and Define.xml. Subsequently, it is necessary to create statistical analysis reports based on the Statistical Analysis Plan (SAP) using data that meets these standards. A count of clinical trial reports is contingent upon project complexity, ranging from hundreds to thousands. Therefore, these aforementioned modules, in conjunction with clinical trials, constitute a primary and critical work task for statistical analysis teams of the pharmaceutical manufacturers and Contract Research Organization (CRO) companies in addressing submission requirements.

Accordingly, there is a desire to provide a method, device, and a storage medium for generating a report from clinical data that can efficiently implement automated report generation from the clinical data.

### SUMMARY

One or more embodiments of the present disclosure provide a method, a device, and a medium for generating a report from clinical data to solve the following technical problem: how to efficiently implement automated report generation from the clinical data.

One or more embodiments of the present disclosure provide a method for generating a report from clinical data. The method includes: via a reporting generation platform, constructing a reporting generation project corresponding to clinical data to be processed and assigning corresponding roles and permissions to processing personnel. The processing personnel include statistical analysts, reviewers, and annotation specialists. The method includes: constructing, by the statistical analysts, a template pending review based on a clinical trial design and publishing the template pending review. The method includes: performing, by the reviewers, online review on the template pending review and add online collaborative annotation, enabling the statistical analysts to obtain a template pending application by updating the template pending review based on the online collaborative annotation. The method includes: performing, by the annotation specialists, template annotation on the template pending application, enabling the reporting generation platform to automatically generate a corresponding SAS program based on the template annotation. The method includes: performing the SAS program to obtain a report pending application.

One or more embodiments of the present disclosure provide a device for generating a report from clinical data. The device includes: at least one processor, and a storage device communicatively connected to the at least one processor. The storage device stores instructions executed by the at least one processor, and the at least one processor is configured to execute the method for generating the report from the clinical data.

One or more embodiments of the present disclosure provide a non-transitory computer storage medium for generating a report from clinical data storing instructions, the instructions, when executed by at least one processor, causing the at least one processor to implement the method for generating the report from the clinical data.

### BRIEF DESCRIPTION OF THE DRAWINGS

The drawings described herein are provided for further understanding of the present disclosure and constitute a part of the present disclosure. The schematic embodiments and descriptions of the present disclosure are used to explain the present disclosure and do not constitute an improper limitation of the present application. In the FIGs:

FIG. 1 is a flowchart of a method for generating a report from clinical data according to some embodiments of the present disclosure.

FIG. 2 is a flowchart of an exemplary process for a statistical logic verification according to some embodiments of the present disclosure.

FIG. 3 is a flowchart illustrating an exemplary process for analyzing template annotation according to some embodiments of the present disclosure.

FIG. 4 is a flowchart of an exemplary process for generating a verification result according to some embodiments of the present disclosure.

FIG. 5 is a schematic diagram illustrating an internal structure of a device for generating a report from clinical data according to some embodiments of the present disclosure.

### DETAILED DESCRIPTION

To make the objectives, technical solutions, and advantages of the present disclosure clearer, the technical solutions of the present disclosure will be described clearly and completely below in conjunction with specific embodiments and corresponding drawings of the present disclosure. Obviously, the described embodiments are only a part of the embodiments of the present disclosure, and not all of them. Based on the embodiments in the present disclosure, all other embodiments obtained by those skilled in the art without making creative efforts fall within the protection scope of the present disclosure.

Statistical analysis work is performed on data collected according to a clinical research protocol. Simply put, the most basic work content is to use the Statistical Analysis System (SAS). The SAS is a professional statistical analysis software recommended in the life sciences field, with all its computational modules certified. This software generates CDISC-compliant datasets (e.g., SD™ and ADaM) required by regulatory authorities and hundreds of reports for the preparation and submission of the Clinical Study Report (CSR).

Before submission, designing report styles and generating statistical analysis reports based on the SAP constitutes a critical task for statisticians. Consequently, statistical analysis teams of pharmaceutical manufacturers and CRO companies must dedicate substantial manpower and resources to this endeavor.

The following problems exist:

1. Report complexity: Reports often undergo frequent revisions to content and format based on different clinical trials or different phases of a trial. The reports contain a large amount of data and statistical analysis results. During the production and review process of the reports, multiple generations and updates may be required based on actual data. Furthermore, modifications may necessitate reverting to amend underlying datasets if initial preparations are inadequate. This complicates the report finalization process, requiring the statistical analysis teams to dedicate considerable time and effort.

2. Data quality issues: Data quality is crucial for the quality and accuracy of the report. However, in practice, issues such as data errors, missing data, and data inconsistencies can slow down the report generation process and even affect the final result.

3. Communication and coordination: Report generation involves a plurality of departments and teams, including data management, statistical analysis, medical writing, or the like. Therefore, communication and coordination work become crucial. Any inadequacy may lead to non-compliant report design, excessive resource consumption, and consequently, compromised report quality.

4. Time pressure: Clinical studies are typically bound by stringent timelines. Consequently, the statistical analysis teams are required to complete the report generation within compressed schedules, which may result in extended work hours for team members and increased work stress.

5. Compliance and quality control: As a critical component of CSR, the reports are required to adhere to all applicable regulatory requirements and guidelines.

Therefore, statistical analysis teams need to focus on compliance and quality control during the report production process to ensure the accuracy and reliability of the report.

One or more embodiments of the present disclosure provide a method, device, and a medium for generating a report from clinical data to solve the following technical problem: how to efficiently implement automated report generation from the clinical data.

The technical solution proposed by the embodiments of the present disclosure is described in detail below with reference to the drawings.

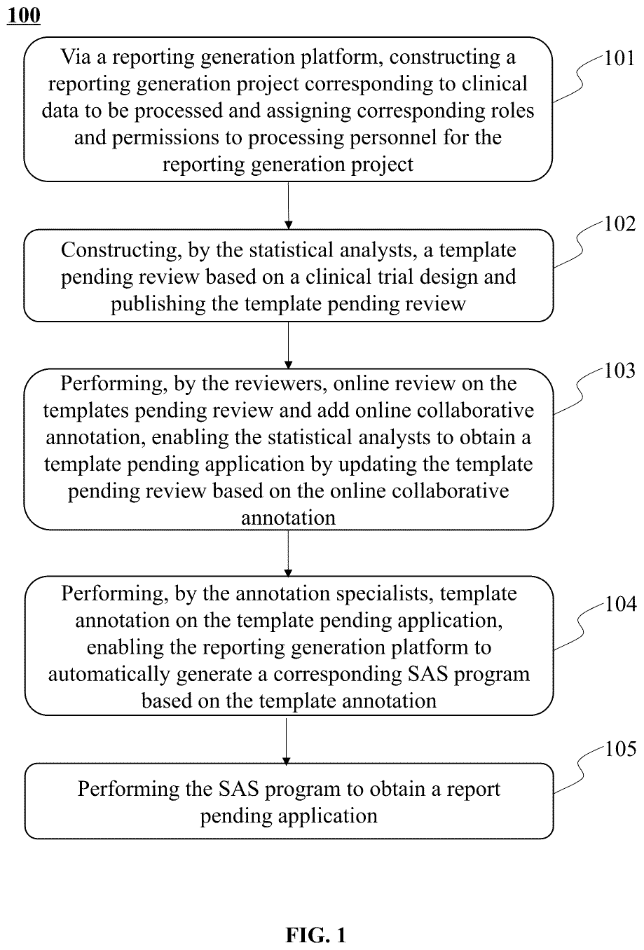

FIG. 1 is a flowchart of a method for generating a report from clinical data according to some embodiments of the present disclosure. The clinical data may also be referred to as clinical trial data. The method for generating a report may also be referred to as a method for generating a clinical trial report. In some embodiments, a process 100 may be executed by a processor within a reporting generation platform. As shown in FIG. 1, the process 100 includes the following operations.

In 101, via the reporting generation platform, a reporting generation project corresponding to clinical data to be processed is constructed and corresponding roles and permissions are assigned to processing personnel for the reporting generation project.

The reporting generation platform refers to an integrated system or online collaborative environment specifically designed to implement automated, standardized, and collaborative production of the clinical trial report. For example, the reporting generation platform is a web-based system for generating the clinical trial report. The platform may provide functional modules such as a user management module, a project creation module, a template design module, a collaborative review module, a program generation module, and a version management module. The reporting generation platform may also be referred to as a clinical trial reporting generation platform.

The clinical data to be processed refers to a raw or intermediate dataset that is standardized, formatted, and waiting to be analyzed to generate a specific clinical trial report. For example, the clinical data to be processed may be a raw dataset compliant with CDISC standards, or the clinical data to be processed may be intermediate analysis data that has undergone preliminary processing. The clinical data to be processed may also be referred to as clinical trial data to be processed.

The clinical trial data refers to a collection of raw data or processed data collected according to a clinical research protocol during a clinical research process, used for statistical analysis and evaluation of the safety and efficacy of a drug or medical device. For example, the clinical trial data includes patient demographics data, laboratory test results, adverse event records, efficacy endpoint indicators, or the like.

The reporting generation project refers to a centralized, structured management unit created for processing the clinical trial data and producing a compliant the clinical trial report. For example, the reporting generation project may be used to organize and manage resources, personnel, data, and processes related to generating all reports for a particular clinical trial research. The reporting generation project may also be referred to as a clinical trial reporting generation project.

In some embodiments, to implement the method for generating the clinical trial report from the clinical data, a user first needs to create the reporting generation project corresponding to the clinical data to be processed via the reporting generation platform. For example, the user may upload the clinical data to be processed to the reporting generation platform. The reporting generation platform verifies the clinical data to be processed and then creates a new project record in the database of the reporting generation platform.

Merely by way of example, the reporting generation platform may be a software system based on a network architecture, to provide the user with a graphical user interface. The reporting generation platform may receive project configuration information input by the user, and create and manage a corresponding reporting generation project based on the project configuration information. The project configuration information may include a project name, a project description, an associated clinical trial data identifier, a list of related processing personnel, or the like.

After the application to create the reporting generation project is approved, the assignment of the roles and permissions for the processing personnel may be completed by a project administrator or the user with corresponding permissions operating on the reporting generation platform. For example, the user may add members to the reporting generation project and configure different roles for different members from a predefined role library of the reporting generation platform. The predefined role library refers to a database storing a plurality of roles and task permissions corresponding to the roles. Different roles have different task permissions, which can prevent irrelevant personnel from making erroneous operations. The task permissions include, but are not limited to: creating or editing report templates, reviewing and annotating report templates, annotating report templates, executing programs, viewing project results, or the like.

The processing personnel refer to professionals within the reporting generation project who collaborate to complete the output of statistical reports from the clinical trial data. The processing personnel may also be referred to as related processing personnel. The processing personnel include: statistical analysts, reviewers, and annotation specialists.

The statistical analysts refer to professionals responsible for designing, constructing, and updating a statistical report template based on the clinical trial protocol and the statistical analysis plan (SAP). For example, the statistical analysts use platform tools to construct the initial report template and modify the initial report template based on review comments.

The reviewers refer to professionals responsible for performing online review, proposing modifications, and providing approval on the report template constructed by the statistical analysts. For example, the reviewers include medical experts, statisticians, and compliance experts. The reviewers may add online collaborative annotation to the templates.

The annotation specialists refer to professionals responsible for converting visual styles and statistical requirements of an approved final report template into machine-readable instructions, enabling the reporting generation platform to construct a corresponding SAS program. As an example, the annotation specialist can annotate a statistical test manner and a model used in a “P value” result cell of the report.

In 102, by the statistical analysts, a template pending review is constructed based on a clinical trial design and the template pending review is published.

The clinical trial design may include elements such as a study objective, a population, a treatment manner, an evaluation indicator, a randomization manner, a blinding manner, and a statistical analysis manner.

The template pending review is a report template initially created by the statistical analysts based on the clinical trial design and is not yet finalized. As an example, the template pending review may be a report template that includes preset statistical tables, listings, and figures, but with specific data being blank. The template pending review may also be referred to as a report template pending review.

In some embodiments, the statistical analysts may construct and publish the template pending review based on an analysis result of the clinical trial design. After the template pending review is published, the reporting generation platform sends a notification to the reviewers to remind the reviewers to perform subsequent operations.

In some embodiments, the constructing, by the statistical analysts, the template pending review based on the clinical trial design includes: determining a keyword pending search based on the clinical trial design, and reading a report template library based on the keyword pending search to determine whether a relevant template whose matching degree against the keyword pending search exceeds a preset threshold exists with the report template library. In some embodiments, the constructing, by the statistical analysts, the template pending review based on the clinical trial design includes: in response to the existence of the relevant template whose the matching degree against the keyword pending search exceeds the preset threshold within the report template library, adjusting the relevant template based on the clinical trial design to obtain the template pending review. In some embodiments, the constructing, by the statistical analysts, the template pending review based on the clinical trial design includes: in response to the existence of the relevant template whose the matching degree against the keyword pending search is less than the preset threshold within the report template library, generating a shell template and constructing the template pending review based on the clinical trial design.

The keyword pending search refers to a word or a phrase that represent a core feature and a statistical requirement of the current clinical trial. As an example, the keyword pending search may include a trial design type (e.g., “randomized”, “double-blind”, etc.), a study population (e.g., “non-small cell lung cancer”), or the like.

In some embodiments, the statistical analyst may extract and determine the keyword pending search from design documents, such as a clinical trial protocol and a statistical analysis plan. As an example, the statistical analysts may parse uploaded documents such as the SAP by using the reporting generation platform to identify a potential keyword. The statistical analysts finally confirm and select a keyword identified by the reporting generation platform as the keyword pending search based on the clinical trial protocol and the statistical analysis plan, or manually input a more precise keyword pending search as a supplement.

The report template library refers to a database that centrally stores, indexes, and manages historical templates and metadata. For example, the report template library may include an efficacy report template, a pharmacokinetics report template, or the like. As another example, the report template library may include a plurality of standard report templates classified by a therapeutic area (e.g., oncology and cardiovascular) or an analysis type (e.g., safety and efficacy). The report template library may also be referred to as a report template knowledge library.

In some embodiments, the reporting generation platform queries and reads the report template library by using a corresponding retrieval and matching algorithm.

The preset threshold is a critical value standard set by the user or the processing personnel, or defaulted by the reporting generation platform, and is used to determine whether the matching degree is sufficient. The preset threshold may be a number from 0 to 1 (including 0 and 1), for example, the preset threshold is 0.7.

The relevant template refers to a historical template associated with the keyword pending search of the template pending review. For example, the relevant template may be a historical template that includes the keyword pending search “baseline”.

In some embodiments, after the processing personnel input the keyword pending search into the reporting generation platform, the reporting generation platform compares the keyword pending search with the metadata and content features of the historical templates in the report template library.

Merely by way of example, the reporting generation platform represents both the historical templates and the keyword pending search as vectors in a multi-dimensional space, obtains a matching degree between 0 and 1 by calculating an indicator such as a cosine similarity between the two vectors. The reporting generation platform determines a historical template with a matching degree greater than the preset threshold (e.g., 0.7) as the relevant template.

In some embodiments, two cases are defined: where the relevant template, whose the matching degree against the keyword pending search exceeds the preset threshold, exists in the report template library, and a case where no such the relevant template exists. The reporting generation platform sorts the historical templates in the report template library in descending order of the matching degrees, and outputs all historical templates with the matching degree being greater than the preset threshold (e.g., 0.7) as the relevant templates. The processing personnel perform a more refined adjustment on the basis of the relevant templates to obtain the template pending review. The adjustment includes modifying a title or a footnote, adding or deleting a statistical indicator, adjusting a grouping variable, updating a data source, changing a format, or the like.

The shell template refers to a basic report template generated when no historical template matching the clinical trial exists within the report template library. For example, the shell template may be a completely blank template.

In some embodiments, in response to the existence of the relevant template whose the matching degree against the keyword pending search is less than the preset threshold within the report template library, the reporting generation platform generates the shell template. The processing personnel may manually construct the template pending review on the basis of the shell template according to the clinical trial protocol and the statistical analysis plan.

In the embodiments provided in the present disclosure, all text and formats on the template pending review are parsed by cross-platform computer programming language and the SAS and are finally completely presented on a final clinical trial report. The text and formats include a title, a footnote, a column header, display text of each paragraph, a blank line between paragraphs, a presentation of a number of decimal places, or the like. These will be fully presented on a final clinical trial report, ensuring that the presentation during report generation, review or modification is consistent with the final presentation. The reporting generation platform may also export the clinical trial report in other formats (e.g., Word and PDF) to meet different requirements, such as project team filing.

In some embodiments, the statistical analysts may publish the template pending review.

The term ‘publish’ refers to an operation in which the statistical analysts submit and push a constructed or modified template pending review to the reviewers via the reporting generation platform, thereby initiating a review process.

In the present embodiment, a mechanism of “keyword retrieval” and “report template library matching” is used to quickly locate the relevant template within the report template library, significantly improving efficiency and standardization of clinical trial report construction.

In 103, by the reviewers, online review on the template pending review and add online collaborative annotation are performed, enabling the statistical analysts to obtain a template pending application by updating the template pending review based on the online collaborative annotation.

The term ‘online review’ refers to an interactive process in which the reviewers connect to and access the reporting generation platform in real time, and directly view, comment on, and propose modifications for the template pending review on the reporting generation platform. For example, the reviewers view the template on a client of the reporting generation platform. The operations of the online review (e.g., viewing, commenting, and proposing modifications) are synchronized in real time to related collaborative personnel.

The online collaborative annotation refers to an electronic comment, question, or modification added by the reviewers directly to a specific position (e.g., a title, a result cell, and a paragraph) of the template pending review on the reporting generation platform. For example, the online collaborative annotation may be a text comment, a highlight mark, or an arrow indication.

In some embodiments, after the statistical analysts publish the template pending review, the reviewer performs the online review on the template pending review and adds the online collaborative annotation.

In some embodiments, the reporting generation platform generates and saves a communication log when the reviewers perform online review on the template pending review and the report pending application and adds the online collaborative annotation, so that the corresponding information can be retrieved and viewed when needed.

The communication log refers to a record of all online collaborative activities generated and saved by the reporting generation platform. The communication log includes a timestamp of the online collaborative annotation (e.g., a date and a time when the online collaborative annotation occurs), an operation role (e.g., the processing personnel who perform the online collaborative annotation), an operation content (e.g., a text content of the annotation), a status change (e.g., a status of the annotation changes from “new” to “resolved”), or the like.

In some embodiments, the reporting generation platform may record related data of the online collaborative annotation performed by the reviewers on the template pending review, generate the communication log based on the related data of the online collaborative annotation, and save the communication log. The related data includes the timestamp, the operation role, the operation content, and the status change of the online collaborative annotation.

The template pending application refers to a report template formed after the template pending review undergoes the online review by the reviewers, and modification and update by the statistical analysts, and is waiting to be applied. For example, the template pending application is a final version table style document that has been approved by all the reviewers. The template pending application may also be referred to as a report template pending application.

In some embodiments, the reviewers may perform the online review on the template pending review and add comments or modifications in real time. The reporting generation platform synchronously updates and performs feedback in real time (e.g., by sending an email and a Feishu message) to the statistical analysts. The statistical analysts may reply to the online collaborative annotation and update the template pending review based on the text content of the online collaborative annotation to obtain the template pending application.

This manner of online collaboration and updating eliminates the need for confirmation via back-and-forth emails, thereby significantly reducing communication overhead among staff members

In some embodiments, to implement batch rapid updating of report templates, the present disclosure may also label different character fields in a plurality of similar templates pending review or a plurality of similar templates pending review when generating the template pending application. The present disclosure determines any one of the plurality of similar templates pending reviews or the plurality of similar templates pending application as an operational template to be processed, and designates all other templates as synchronized templates. During modification of the operational template to be processed, the present disclosure triggers an update component of the synchronized templates corresponding to the operational template to be processed to complete updates for the corresponding synchronized templates.

The different character fields refer to a specific area or cell where content, meaning, or a data source differs among a plurality of similar templates. For example, in similar “template a”, “template b”, and “template c”, the “template a” additionally includes a “visit point” compared to the “template b” and the “template c”. The “visit point” is a different character field of the “template a”.

In some embodiments, the reporting generation platform may determine whether structural frameworks of the plurality of templates pending review or the plurality of templates pending application are consistent based on underlying structures (e.g., a table framework, a title hierarchy, a paragraph style, etc.) of the plurality of templates pending review or the plurality of templates pending application. In response to a determination that the structural frameworks of the plurality of templates pending review or the plurality of templates pending application are consistent, whether public texts (e.g., an equation, a statistical term, etc.) in cells or paragraphs of the plurality of templates pending review or the plurality of templates pending application are consistent is determined. In response to a determination that the public texts (e.g., the equation, the statistical term, etc.) in the cells or the paragraphs of the plurality of templates pending review or the plurality of templates pending application are consistent, the plurality of templates pending review or the plurality of templates pending application are determined to be similar.

In some embodiments, there are two known cases regarding the structural frameworks of the plurality of templates pending review or the plurality of templates pending application: consistent and inconsistent. In response to a determination that the structural frameworks of the plurality of templates pending review or the plurality of templates pending application are consistent, whether the public texts in the cells or paragraphs of the plurality of templates pending review or the plurality of templates pending application are consistent is determined. In response to a determination that the structural frameworks of the plurality of templates pending review or the plurality of templates pending application are inconsistent, the plurality of templates pending review or the plurality of templates pending application are determined to be dissimilar.

In some embodiments, there are two known cases regarding the public texts in the cells or paragraphs of the plurality of templates pending review or the plurality of templates pending application: consistent and inconsistent. In response to a determination that the public texts in the cells or paragraphs of the plurality of templates pending review or the plurality of templates pending application are consistent, the plurality of templates pending review or the plurality of templates pending application are determined to be similar. In response to a determination that the public texts in the cells or paragraphs of the plurality of templates pending review or the plurality of templates pending application are inconsistent, the plurality of templates pending review or the plurality of templates pending application are determined to be dissimilar.

In some embodiments, the reporting generation platform may identify and label the different character fields in the template pending review or the template pending application based on structural comparison, content comparison, semantic analysis, and data source analysis of the template pending review or the template pending application.

The operational template to be processed refers to a template, among a plurality of similar templates, that is selected and directly modified by the processing personnel. The operational template to be processed may also be referred to as an operational report template to be processed.

The synchronized templates refer to other template instances, among the plurality of similar templates except the operational template to be processed, that are labelled with the different character fields and need to be automatically updated based on modifications of the operational template to be processed. For example, in the similar “template a”, “template b”, and “template c”, the “template a” serves as the operational template to be processed, the “template b” and the “template c” serve as the synchronized templates. The synchronized template may also be referred to as a synchronized report template.

In some embodiments, the operational template to be processed may be specified by the user or the processing personnel.

The update component refers to a software function module or a program unit that implements template synchronous updating. For example, the update component may include an automated script, a rule engine, or the like, built into the reporting generation platform.

In some embodiments, the update component is configured to monitor changes of the operational template to be processed. In response to a determination that detects a change of the operational template to be processed, the update component is triggered and performs the following steps: identifying modification content of the operational template to be processed; locating a character field in the synchronized template that is the same as a character field in the operational template to be processed; applying the modification content of the operational template to be processed corresponding to the character field that is the same in the synchronized template and the operational template to be processed to a corresponding character field of the synchronized template.

In some embodiments, when the update component detects the change of the operational template to be processed, there are two known cases: detecting the change of the operational template to be processed and detecting no change of the operational template to be processed. In response to a determination that detects the change of the operational template to be processed, the update component performs the aforementioned steps of updating the synchronized template.

For example, the update component recognizes that the modification content of “operational template to be processed a′” involves the “age” character field. “synchronized template b” and “synchronized template c′” have the same character fields as the “operational template to be processed a′”, including the “age” character field and the “baseline” character field. The update component applies the modification content involving the “age” character field of the “operational template to be processed a” to the “age” character field of the “synchronized template b′” and the “synchronized template c′”, that is the same as the “operational template to be processed a′”.

In the present embodiment, by labelling differences among the plurality of similar templates, a template group with linked updates is constructed. When the processing personnel modify the character field that is the same in the operational template to be processed and the synchronized templates, the change can be automatically and accurately synchronized to all synchronized templates. Unique character fields of the template group are protected. Thus, a large number of similar templates are maintained efficiently and reliably.

In 104, by the annotation specialists, template annotation on the template pending application is performed, enabling the reporting generation platform to automatically generate a corresponding SAS program based on the template annotation.

The template annotation refers to machine-readable or machine-parsable annotations made on the template pending application for fields (parameter report fields) that require content to be automatically generated by a program. The annotations describe calculation logic, a data source, or a display format of the fields. For example, the template annotation may be an instruction pointing to a specific variable in a specific analysis dataset, or a macro call containing a statistical equation.

In some embodiments, after obtaining the template pending application, the annotation specialists perform the template annotation on the template pending application firstly.

In some embodiments, the performing, by the annotation specialists, the template annotation on the template pending application includes: determining a parameter field to be annotated in the template pending application, and performing programmatic annotation or natural language annotation on the parameter field to be annotated as to generate a target annotation; in response to determining that a logic verification function is triggered, performing logic verification on the target annotation to determine whether the target annotation complies with a writing standard; and in response to determining that the target annotation complies with the writing standard, completing the template annotation on the template pending application.

The parameter field to be annotated refers to a specific area or cell in the template pending application that needs to be replaced by calculated data or a statistical result. For example, the parameter field to be annotated may be a cell where the “average age” to be replaced by the statistical result is located. The parameter field to be annotated may also be referred to as a parameter report field to be annotated.

The programmatic markup annotation refers to using a structured, programming language-like grammar or a specific rule to provide explanations for the parameter field in the template. For example, the programmatic annotation may be an annotation based on the SAS language.

The natural language annotation refers to using an everyday language expression to provide explanations for the parameter field in the template. For example, the natural language annotation may be “calculate the average value of age”.

In some embodiments, the target annotation is a programmatic or natural language annotation performed by the annotation specialists on the parameter field to be annotated after the annotation specialists determining the parameter field to be annotated in the template pending application. For example, the annotation specialists may add the target annotation via a template editing and review interface integrated into the reporting generation platform. The target annotation includes all annotations added by all annotation specialists on the template pending application.

The logic verification refers to a series of syntax and logic checks performed by the reporting generation platform on the target annotation added by the annotation specialists to ensure that the target annotation can be correctly parsed. For example, the logic verification function checks whether a macro function name is correct, whether parameters are complete, whether a variable name exists in a specified dataset, or the like.

The writing standard refers to a predefined, structured syntax rule to ensure that a template standard can be parsed and executed by a machine. For example, the writing standard includes a syntax format, a semantic rule, a logic rule, or the like.

In some embodiments, the reporting generation platform may use a parser generator to predefine syntax rules of an annotation language. For example, the parser generator is Another Tool for Language Recognition (ANTLR). After syntax analysis generates an Abstract Syntax Tree (AST), the reporting generation platform traverses the AST and performs context-related checks. In some embodiments, the reporting generation platform may use a trained natural language processing model to map elements extracted by the natural language processing model to programmatic instructions. The programmatic instructions are used to check the natural language annotation.

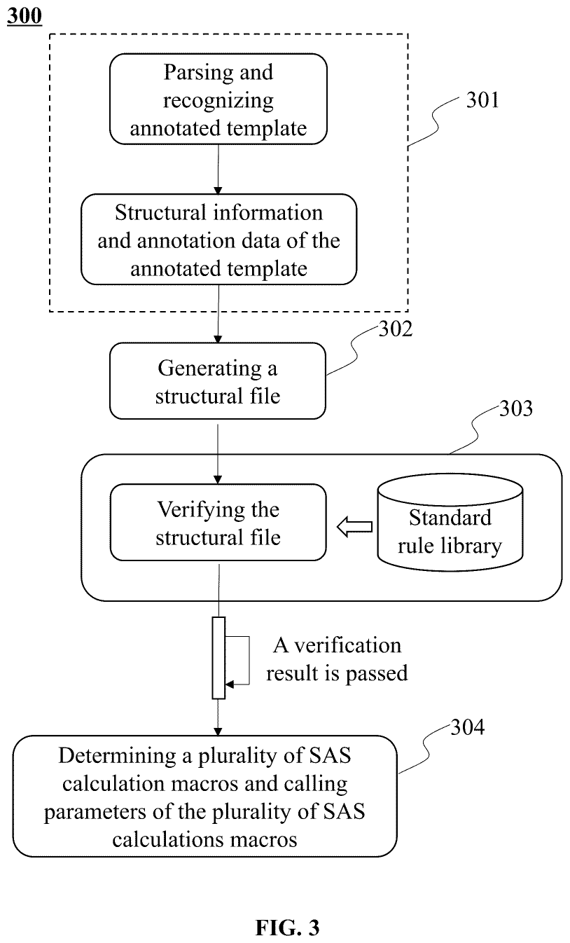

More descriptions regarding the logic verification may be found elsewhere in the present disclosure (e.g., FIG. 3 and related descriptions thereof).

In the present embodiment, by triggering the logic verification after performing the template annotation, errors in the template annotation are detected and corrected in a timely manner. This ensures the accuracy and reliability of the conversion from the template annotation to the SAS program. Furthermore, allowing the template annotation to be performed in different forms enables the annotation specialists with varying levels of proficiency to produce high-quality template annotation that can be correctly parsed by the machine.

In some embodiments, the reporting generation platform may parse the template annotation to convert the template annotation into a plurality of SAS calculation macros and calling parameters of the plurality of SAS calculation macros.

In some embodiments, the reporting generation platform constructs the corresponding SAS program based on the template annotation.

In some embodiments, the reporting generation platform analyzes the template annotation based on cross-platform computer programming language and a SAS parsing engine to convert the template annotation into the plurality of SAS calculation macros and the calling parameters of the plurality of SAS calculation macros. The reporting generation platform summarizes the plurality of SAS calculation macros and the calling parameters of the plurality of SAS calculation macros to obtain the corresponding SAS program.

In some embodiments, the reporting generation platform uses R language or the cross-platform computer programming language instead of the SAS to perform core operations related to statistical analysis.

The calling parameters refer to specific values or variables transmitted to the SAS calculation macros. These parameters are used to indicate key information required for the macro to perform calculations. In some embodiments, the calling parameters include parameters such as specified datasets, specified analysis variables, specified grouping variables, specified statistical manners, etc. The SAS calculation macro and the calling parameter of the SAS calculation macro may also be referred to as a SAS core calculation macro program and parameter corresponding to automatic filling of the macro.

In some embodiments, the reporting generation platform converts the template annotation into a plurality of corresponding SAS calculation macros and calling parameters of the plurality of corresponding SAS calculation macros via an analysis library. The analysis library contains a plurality of SAS calculation macros and calling parameters of the plurality of SAS calculation macros. More descriptions regarding the analysis library and converting the template annotation into the plurality of SAS calculation macros and the calling parameters of the plurality of SAS calculation macros may be found elsewhere in the present disclosure (e.g., FIG. 3 and related descriptions thereof).

In some embodiments, the reporting generation platform may generate calling codes corresponding to the plurality of SAS calculation macros based on the plurality of obtained SAS calculation macros and the calling parameters of the plurality of obtained SAS calculation macros. The reporting generation platform may generate the required SAS program based on the generated calling codes. More descriptions regarding the calling codes, and summarizing the plurality of SAS calculation macros and the calling parameters of the plurality of SAS calculation macros to obtain the SAS program, may be found in the related descriptions below.

In some embodiments of the present disclosure, automated technical means efficiently, accurately, and standardly convert the template annotation into an executable SAS program. This overall improves the generation efficiency, accuracy, and standardization level of the report.

In some embodiments, after analyzing, via the reporting generation platform, the template annotation based on the cross-platform computer programming language and the SAS parsing engine, further includes: analyzing the clinical data to be processed to parse the clinical data to be processed into variable units of corresponding filling parameters; and summarizing the template annotation and the variable units to obtain a required dataset documentation.

In some embodiments, after the SAS parsing engine analyzes the template annotation, the clinical data to be processed is also parsed by the reporting generation platform into the variable units. The reporting generation platform summarizes the variable units and all annotations within the same project. Finally, the reporting generation platform automatically generates the required dataset documentation based on these summarized results, combined with CDISC standards. The required dataset documentation includes all dataset and variable information required to generate the report. Manual creation of a blank description file and manual entry of variables one by one are not required.

The SAS parsing engine refers to a software module with parsing functionality that is integrated into the reporting generation platform.

In some embodiments, the SAS parsing engine is configured to parse the template annotation and convert the statistical intents and parameters expressed by these annotations into the required SAS calculation macro and the calling parameter of the SAS calculation macro.

The corresponding filling parameter refers to an automated matching relationship and data interface specification. The corresponding filling parameter is used to implement matching between the SAS calculation macro and the corresponding data. For example, during the process of generating the SAS program, based on the annotation content, the reporting generation platform automatically calls statements for the SAS core computation macro program, to fill in specific information required for the performing the SAS core computation macro program. The specific information includes actual variable values or dataset names. In some embodiments, the corresponding filling parameters include parameters such as data source parameters, analysis variable parameters, data filter parameters, statistical manner parameters, or the like.

In some embodiments, the data source parameter refers to a parameter that causes the SAS calculation macro to determine a specific dataset. The analysis variable parameter refers to a parameter that causes the SAS calculation macro to determine a variable. The data filter parameter refers to a parameter that causes the SAS calculation macro to determine a filtering manner before data analysis. The statistical manner parameter refers to a parameter that causes the SAS calculation macro to determine a statistical calculation manner.

The variable unit refers to a structured information entity formed after analyzing and extracting the clinical data to be processed. The variable unit is used to describe data characteristics. In some embodiments, the variable unit includes key attribute information such as a variable name, a label, a type, a value, or the like.

In some embodiments, after parsing the clinical data to be processed, the reporting generation platform reads metadata of the clinical data to be processed and converts the data into a structured variable unit object. Subsequently, the reporting generation platform performs matching processing between these variable units and parameters required for the plurality of SAS calculation macros (i.e., the corresponding filling parameters). This ensures that the automatically generated SAS program can accurately call the correct data source and variables to perform calculations.

The required dataset documentation refers to a structured metadata summary file automatically generated by the reporting generation platform. The required dataset documentation defines the source, derivation rules, and purpose of each variable in an analysis dataset constructed for performing a specific statistical analysis. The required dataset documentation may include data such as the statistical manner used, the data source, and data filter conditions. The required dataset documentation may also be referred to as an ADaM analysis dataset derivation document.

In some embodiments, the reporting generation platform intelligently matches and associates the statistical intents parsed from the template annotation with the variable units of the corresponding filling parameters parsed from the clinical data to be processed. All data mapping relationships are encapsulated into a structured and traceable required dataset documentation.

Some embodiments of the present disclosure implement precise, automated between data and programs by automatically parsing the clinical data and generating a structured document. This improves efficiency while ensuring the traceability and compliance of the entire report generation process.

In 105, the SAS program is performed to obtain a report pending application.

The report pending application refers to a report whose format and logic are finalized but not yet filled with the clinical data. The report pending application may also be referred to as a clinical trial report pending application.

In some embodiments, after constructing the corresponding SAS program, the reporting generation platform performs the SAS program to obtain the report pending application.

Merely by way of example, the reporting generation platform may configure environment parameters required for running the SAS program (e.g., a library path, a macro variable, etc.). The reporting generation platform may submit the SAS program to a corresponding server or grid environment for performing, waiting to be triggered. After the SAS program is triggered, the reporting generation platform continuously monitors a performance status (e.g., the SAS program is running, successfully completed, or failed to run) of the SAS program. After the SAS program is successfully performed, a performance result is generated. For example, the performing result may include a SAS result dataset and the report pending application. More descriptions regarding the SAS result dataset may be found in the related descriptions below.

In some embodiments, after performing the SAS program to obtain the report pending application, the method further includes: storing the report pending application and performing the SAS result dataset generated by the SAS program.

The SAS result dataset refers to one or more dataset files containing final statistical results generated after the reporting generation platform performs the SAS program. The SAS result dataset contains intermediate SAS datasets with all statistical operation results (e.g., a mean, a standard deviation, a P value, etc.). The statistical operation results are used to fill a final report table. In some embodiments, the SAS result dataset may include core statistical result data values such as a mean, a standard deviation, or a confidence interval, and data such as Key variables supporting version comparison.

In some embodiments, the reporting generation platform reads the obtained SAS result datasets and fills the data therein into corresponding cells of the table to obtain the report pending application.

In some embodiments, the reporting generation platform associates and stores the report pending application with the SAS result datasets to facilitate subsequent operations, such as version comparison of the report.

In some embodiments, storing the report pending application and performing the SAS result dataset generated by the SAS program includes: constructing independent Key variables for each of a plurality of result cells in the report pending application, respectively. The Key variables store metadata information of the plurality of result cells, the metadata information includes a row/column coordinate, an input dataset name, a variable name, a variable value or label, and a generation condition. The method further includes: in response to version comparison instructions, matching the Key variables to the corresponding result cells of the report pending applications in different versions and displaying differences in the corresponding result cells.

The result cell refers to a single data unit in the final generated report pending application used to store a statistical calculation result of the SAS program. In some embodiments, content in the result cell may include data such as a mean, a standard deviation, or the like.

The Key variable refers to a unique identifier that the reporting generation platform automatically constructs for each result cell of the report pending application. The identifier is associated with and stores complete metadata information for generating the result cell. In some embodiments, the Key variable may be a unique string. The metadata information of the result cell is stored in the attributes of the Key variable.

In some embodiments, when the report pending application is generated, the reporting generation platform automatically extracts the complete metadata information of each result cell. The reporting generation platform combines the extracted metadata information into a unique string identifier based on a set of predefined naming rules. This set of rules ensures that the Key variables of different result cells are not duplicated. The reporting generation platform binds and stores the unique Key variable with its corresponding result cell.

In some embodiments, the predefined naming rules may be selected as needed. Common naming rules include concatenation rules, hashing rules, and hybrid rules.

The version comparison instruction refers to a trigger signal or command that causes the reporting generation platform to initiate a built-in version comparison function. In some embodiments, the version comparison instruction may be used to compare two or more versions of the report pending application, and display the differences of the result cells corresponding to the different versions of the report pending application.

The differences refer to content differences between the result cells corresponding to the Key variables in different versions of the report pending application. In some embodiments, the differences may include modifications, additions, deletions, or the like.

In some embodiments, after performing the version comparison instruction, the reporting generation platform compares the content differences of the result cells corresponding to the Key variables in different versions of the report pending application. The content differences are presented visually in the report.

In some embodiments, the differences are displayed intuitively in the report and highlighted with colors.

The modifications: Indicate changes in results between different versions. The modifications are marked in red and display the previous result.

The additions: indicating new result rows compared to the comparison version. The additions are marked in green.

The deletions: indicating deleted result rows compared to the comparison version. The deletions are marked in gray and placed at the end.

Some embodiments of the present disclosure construct the independent Key variable carrying the complete metadata for the each result cell. Based on this, the version comparison instruction is performed. This enables automated and precise localization and quantitative analysis of differences between cross-version reports. It replaces inefficient and error-prone manual comparisons and improves the efficiency of clinical data review. Simultaneously, this mechanism provides clear and traceable clues for each data iteration, deeply satisfying regulatory compliance requirements.

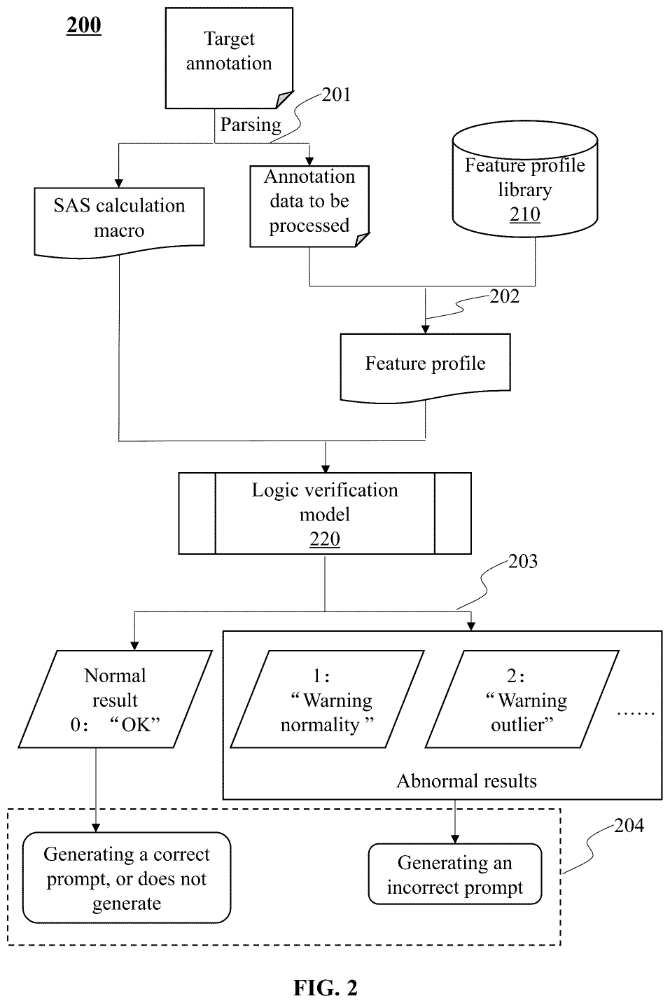

FIG. 2 is a flowchart of an exemplary process for a statistical logic verification according to some embodiments of the present disclosure.

In some embodiments, the logic verification includes grammar logic verification and statistical logic verification. After obtaining the target annotation of the annotation specialists, the reporting generation platform performs the logic verification on the target annotation.

Merely by way of example, after designing the template pending application, the annotation specialists may add the target annotation for the template according to a preset annotation rule to tells an Agent: “What content should be placed in this cell?”. After the annotation specialists have completed all required annotations, an annotated file is obtained. The reporting generation platform performs the grammar logic verification and the statistical logic verification.

The Agent is an automated computer program. The Agent may be written and obtained according to requirements, enabling it to autonomously perform tasks.

The grammar logic verification refers to verifying whether the template annotation complies with a grammar standard. For example, the logic verification includes verifying whether there are clerical errors in the template annotation. In some embodiments, the reporting generation platform may be configured to trigger the grammar logic verification when the processing personnel complete the input of a template annotation and then lose focus (e.g., after the processing personnel finish writing the template annotation in a Word comment box and then click elsewhere on the template).

The statistical logic verification refers to verifying whether the relationship between the annotation data to be processed and the SAS calculation macro complies with a statistical principle. For example, the use of a SAS calculation macro for a t-test on clinical data with a severely skewed distribution may yield inaccurate results. The annotation data to be processed refers to clinical data specified by the target annotation and used for the statistical analysis. For example, if the target annotation is “calculate the mean and standard deviation of ‘AGE’”, then the annotation data to be processed is the “AGE” variable. In some embodiments, the reporting generation platform may be configured to trigger the statistical logic verification by the processing personnel clicking a button on the client of the reporting generation platform.

As shown in FIG. 2, the process 200 of the statistical logic verification includes following operations.

In 201, annotation data to be processed corresponding to the target annotation and SAS calculation macro corresponding to the target annotation are determined by analyzing the target annotation.

In some embodiments, a reporting generation platform may perform format parsing and annotation data recognition on the target annotation to obtain structural information and annotation data of the target annotation. Based on the structural information and the annotation data, the reporting generation platform generates a structural file corresponding to the target annotation. The reporting generation platform matches and determines the SAS calculation macro corresponding to the target annotation based on the structural file via an analysis library built into the reporting generation platform.

More descriptions regarding parsing the target annotation and determining the annotation data to be processed corresponding to the target annotation and the SAS calculation macro corresponding to the target annotation, may be found in the related descriptions below.

In 202, a feature profile corresponding to annotation data to be processed is retrieved from a feature profile library 210 built into the reporting generation platform based on the annotation data to be processed.

In some embodiments, the reporting generation platform may perform automated exploratory analysis on an ADaM dataset in advance. A feature profile is generated for each variable and cached in the feature profile library 210 built into the reporting generation platform.

The variable refers to each column in the ADAM dataset. The ADAM dataset is typically a two-dimensional table. Each column represents a variable. For example, in Analysis DataSet Subject Level (ADSL): “USUBJID” is a column representing a unique subject identifier; “AGE” is a column representing age; “SEX” is a column representing gender; “TRT01P” is a column representing an actual treatment group. The “USUBJID”, the “AGE”, the “SEX”, and the “TRT01P” are all variables.

The feature profile refers to a structured, quantified statistical feature summary of a variable in the ADAM dataset. Merely by way of example, the reporting generation platform traverses each column of the ADAM dataset, and generates the feature profile for each column individually. For example, after processing the “USUBJID” column, the “AGE” column, and the “SEX” column, the reporting generation platform generates a feature profile for the “USUBJID” column, a feature profile for the “AGE” column, and a feature profile for the “SEX” column, respectively. The feature profile may include: distribution shape (e.g., normal distribution, skewed distribution, etc.), outlier information (e.g., a count of outliers detected through a certain manner, such as Interquartile Range rule (IQR)), data sparsity (e.g., a count of unique values), or the like.

In 203, a result of the statistical logic verification is obtained based on the SAS calculation macro and the feature profile.

In some embodiments, the reporting generation platform may obtain the result of the statistical logic verification based on the SAS calculation macro and the feature profile via a logic verification model 220. For example, the reporting generation platform may input the SAS calculation macro and the feature profile into the logic verification model 220. The logic verification model 220 outputs the result of the statistical logic verification.

The result of the statistical logic verification may be a multi-classification result. For example, the result of the statistical logic verification includes 0: “OK” (logically reasonable, no risk); 1: “Warning normality” (warning: the data is not normally distributed, parametric test results may be unreliable); 2: “Warning outlier” (warning: there are significant outliers that may affect the results), etc.

The logic verification model 220 may be obtained by training based on at least one group of training samples with a label. In some embodiments, the training samples may include positive samples and negative samples. The positive sample refers to a sample that uses a correct SAS calculation macro. For example, a T-test is performed on the annotation data to be processed that follows a normal distribution. The label of the positive sample is 0 (indicating normal). The negative sample refers to a sample that uses an incorrect SAS calculation macro. For example, the T-test is performed on the annotation data to be processed that does not follow the normal distribution. The label of the negative sample is manually marked according to an error type, e.g., the label of the negative sample is 1 or 2.

During training, the training samples are input into an initial logic verification model, and a loss function is constructed based on the output of the initial logic verification model and the label. Parameters of the initial logic verification model are iteratively updated based on the loss function until the parameters of the initial logic verification model satisfy a preset training condition, then the training is completed. A trained prediction model is obtained, and the trained logic verification model is designated as the logic verification model 220. The preset training condition may include, but is not limited to, the loss function converging, the training cycle reaching a threshold, or the like.

In 204, a prompt of the target annotation is generated based on the result of the statistical logic verification.

In some embodiments, the reporting generation platform may generate the prompt based on an output of the logic verification model 220. For example, if the output of the logic verification model 220 is 0 (indicating normal), the reporting generation platform may generate a correct prompt, or may not generate. As another example, if the output of the logic verification model 220 is a non-zero value (indicating abnormal), the reporting generation platform may display a corresponding error prompt.

In the present embodiment, the target annotation is verified by combining the grammar logic verification and the statistical logic verification, preventing misuse of a statistical manner (e.g., performing a parametric test on non-normal data). This avoids generating misleading or unreliable statistical conclusions from the source and ensures quality and credibility of a final clinical trial report.

FIG. 3 is a flowchart illustrating an exemplary process for analyzing template annotation according to some embodiments of the present disclosure.

In some embodiments, as shown in FIG. 3, the process 300 of analyzing, via a reporting generation platform, a template annotation based on cross-platform computer programming language and a SAS parsing engine to convert the template annotation into a plurality of SAS calculation macros and calling parameters of the plurality of SAS calculation macros, includes:

In 301, annotated template is parsed and recognized to obtain structural information and annotation data of the annotated template.

The annotated template is a template pending application for which template annotation has been completed.

The structural information refers to metadata and a framework used to define and organize content components of the template and their interrelationships. For example, the structural information is table information and text information in the annotated template. In some embodiments, the structural information includes “positions”, “coordinates”, and “attributes” of various data and elements (e.g., text, tables, cells in tables, pictures, etc.) in the template.

The annotation data refers to machine-readable instructions added to the template by processing personnel to guide the reporting generation platform to generate a statistical result and a report. For example, the annotation data may be Python code.

In some embodiments, the reporting generation platform may perform format parsing on the annotated template to obtain the structural information. Merely by way of example, if the annotated template is an MS Word (.docx) file, the reporting generation platform uses a dedicated library (e.g., python-docx for Python or Apache POI for Java) to decompress the .docx file and read an XML file therein. The XML file defines all content of the annotated template, including paragraphs, tables, cell borders, text fonts, or the like. The reporting generation platform obtains the structural information based on data in the XML file.

As another example, if the annotated template is an HTML file, the reporting generation platform uses a standard HTML parser (e.g., Beautiful Soup or lxml of Python) to convert the HTML file into a tree structure (DOM Tree). The reporting generation platform traverses the tree structure to find elements such as <table>, <tr> (row), <td> (cell), or the like, as the structural information.

In some embodiments, the reporting generation platform may obtain the annotation data based on annotation data recognition. Merely by way of example, if the annotated template is the MS Word (.docx) file, when annotation specialists add a “template comment” to a cell, the content of the template comment is an annotation string in a standard format. The reporting generation platform specifically checks whether each cell (or paragraph) exists an associated “template comment”. If the associated “template comment” exists, the reporting generation platform reads the text of the template comment as the annotation data.

In 302, a structural file corresponding to the annotated template is generated based on the structural information and the annotation data.

In some embodiments, the reporting generation platform may convert the structural information and the annotation data into a machine-readable structured format to obtain the structural file. The structural file refers to a file whose content is organized into a fixed, machine-readable format. For example, the structural file is a JavaScript Object Notation (JSON) array.

In 303, the structural file is verified based on a standard rule library built into the reporting generation platform to generate a verification result.

The standard rule library refers to a knowledge library that integrates a plurality of rules to ensure standardization and accuracy of statistical programming. For example, the standard rule library may integrate a plurality of grammar rules and statistical logic rules. In some embodiments, the reporting generation platform may verify, based on the standard rule library, whether the target annotation of the structural file is compliant in terms of a grammar standard. For example, the reporting generation platform checks whether the target annotation is complete, whether the logic of the target annotation is self-consistent, whether variables and datasets requested by the target annotation exist, or the like.

More descriptions regarding the verification may be found in the related descriptions below.

In 304, in response to the verification result is passed, the plurality of SAS calculation macros and the calling parameters of the plurality of SAS calculation macros are matched and determined based on the structural file and via an analysis library built into the reporting generation platform.

The analysis library refers to a database pre-stored with a large number of modular statistical analysis manners. The analysis library contains a large number of highly encapsulated SAS calculation macros. For example, the analysis library contains a SAS calculation macro for calculating a mean and a standard deviation.

In some embodiments, it is known that there are two cases where the verification result is passed and the verification result is failed. In response to the verification result is passed, the reporting generation platform determines, based on the structural file via the analysis library, the plurality of SAS calculation macros. For example, the reporting generation platform, based on {“statistic”: “Mean”, . . . } in the structural file, matches a SAS calculation macro for calculating an “average statistic” from the analysis library. As another example, the reporting generation platform, based on {“statistic”: “Incidence Rate”, . . . } in the structural file, matches an SAS calculation macro for calculating a frequency and a percentage from the analysis library.

In some embodiments, for each SAS calculation macro, the reporting generation platform extracts a call parameter of the SAS calculation macro from a corresponding position in the structural file. For example, the reporting generation platform may convert natural language or semi-structured instructions in the template annotation into machine-understandable, structured parameter key-value pairs through parsing and matching. The reporting generation platform may use the parameter key-value pairs as the call parameter of the SAS calculation macro.

In the present embodiment, by parsing and recognizing the annotated template and generating the structural file, a precise understanding and digital conversion of unstructured annotation data are implemented. Thereafter, based on the analysis library, it automatically matches and generates an executable SAS program, thereby transforming the report programming task into an efficient and reliable automated process, which greatly enhances the generation efficiency of clinical trial reports. In addition, by introducing the built-in standard rule library to verify the structural file, logic errors, parameter omissions, and format non-compliance in the template annotation can be effectively intercepted and prompted at the front end, ensuring the quality of the template annotation.

In some embodiments, summarizing the plurality of SAS calculation macros and the calling parameters of the plurality of SAS calculation macros to obtain the corresponding SAS program includes:
generating calling codes of at least one SAS calculation macro based on the plurality of SAS calculation macros and the calling parameters of the plurality of SAS calculation macros. In some embodiments, summarizing the plurality of SAS calculation macros and the calling parameters of the plurality of SAS calculation macros to obtain the corresponding SAS program further includes: generating the SAS program based on the calling code corresponding to the at least one SAS calculation macro.

In some embodiments, the reporting generation platform queries a macro metadata database built into the reporting generation platform based on the plurality of SAS calculation macros and the call parameters of the plurality of SAS calculation macros to obtain a code template. The reporting generation platform verifies and completes the calling parameters and injects the call parameters into the code template to generate the calling code. The macro metadata database refers to a database that stores metadata of the plurality of SAS calculation macros. For example, the macro metadata database may be a JSON/XML configuration file.

In some embodiments, the reporting generation platform concatenates the calling code corresponding to the at least one SAS calculation macro, supplements header settings, data processing steps, and output settings of the calling code, to form a complete, executable SAS program.

In some embodiments, the reporting generation platform may run the SAS program and obtain a final clinical trial report based on a performance result of the SAS program. Merely by way of example, an Agent of the reporting generation platform submits the finally generated SAS program to a SAS environment for performing. The performance of the SAS program generates the performance result. The performing result typically includes a SAS result dataset and a report pending application. The Agent obtains the performing result. According to previously parsed structural information (e.g., positions, coordinates of various data and elements in the template, etc.), the Agent accurately backfills the SAS result dataset into corresponding positions of the report pending application (i.e., a Word or HTML template (Shell)). The final clinical trial report is generated after all data is filled. A style of the final clinical trial report is consistent with the style of the report pending application. Blank spaces of the final clinical trial report are filled with real and accurate statistical analysis data.

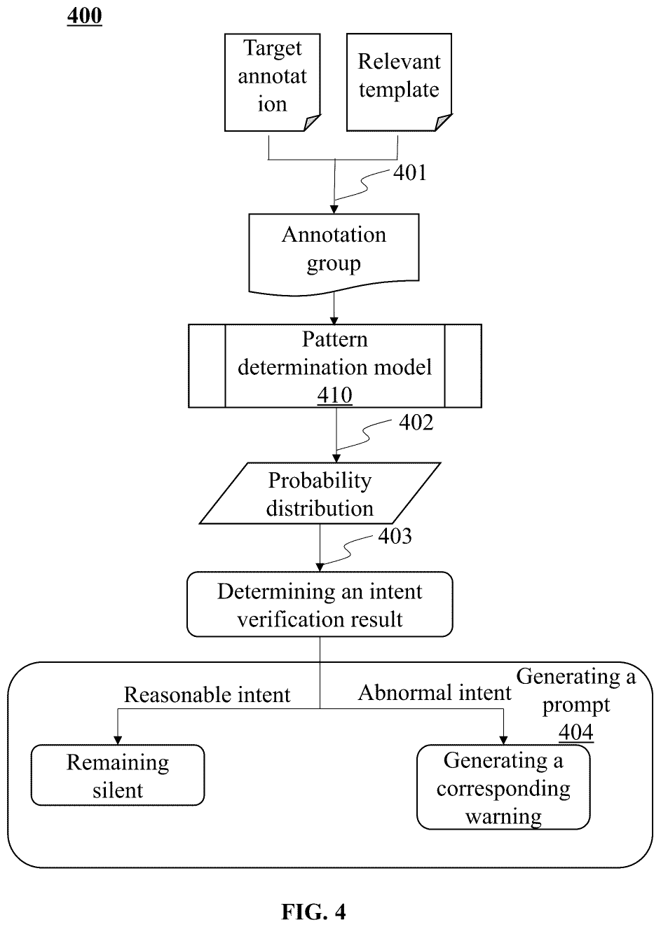

FIG. 4 is a flowchart of an exemplary process for generating a verification result according to some embodiments of the present disclosure. In some embodiments, as shown in FIG. 4, the process 400 of verifying a structural file based on a standard rule library built into a reporting generation platform to generate a verification result includes:

In 401: a plurality of target annotations and association annotations of the plurality of target annotations in the structural file are obtained, and a plurality of annotation groups corresponding to the plurality of target annotations are constructed based on the plurality of target annotations and the association annotations of the plurality of target annotations.

The association annotation refers to an annotation that has a logical or positional association with the target annotation. In some embodiments, the reporting generation platform may determine all annotations within an association range of the target annotation as the association annotations based on the position of the target annotation in the template. The association range may include a table, a chapter, a row, or a column where the target annotation is located. The association range may be set by a user or processing personnel, or defaulted by the reporting generation platform.

For each of the plurality of annotation groups, the reporting generation platform may perform the following operations:

In 402, a probability distribution of a statistical logic pattern for the annotation group is determined based on the annotation group.

In some embodiments, the reporting generation platform may determine the probability distribution of the statistical logic pattern of the annotation group based on the annotation group via a pattern determination model. For example, the reporting generation platform may input the annotation group into the pattern determination model. The pattern determination model outputs a probability distribution vector. The probability distribution vector represents a probability distribution that the statistical logic pattern of the annotation group belongs to various predefined patterns. The predefined pattern is a pre-set definition that outlines common and typical analysis patterns in clinical trial reports. The predefined pattern may include Pattern_Demographics: demographic baseline characteristic analysis (typically includes variables such as “AGE”, “SEX”, “RACE”, with statistics being Mean/Std/N/%), Pattern_Efficacy_Comparison: primary efficacy endpoint comparison (typically compares the same efficacy variable “AVAL” across treatment groups, with statistics being mean, p-value, etc.), Pattern_Safety_Incidence: summary of adverse event incidence (typically grouped by different event types Adverse Events DECOded (AEDECOD) and treatment groups, with statistics being N/%), Pattern_Lab_Shift Table: laboratory test shift matrix (analyzing grade changes of baseline and post-visit), Pattern_Subgroup_Analysis: subgroup analysis (within multiple FILTER conditions, besides the treatment group, there is a common subgroup variable, such as “SEX” or “AGEGR1”), and Pattern_Outlier/Anomaly: outlier/no clear pattern, serving as a “catch-all” category for all patterns that do not fit the above categories.

The pattern determination model 410 may be obtained by training based on at least one group of training samples with a label. In some embodiments, the training samples may include positive samples and negative samples. The positive sample refers to a real and reasonable annotation group. A label vector of the positive sample is (0, 0, 0, . . . , 1, 0, 0). An element value corresponding to the statistical logic pattern of the annotation group is 1 in the label vector. Other element values are 0. The negative sample refers to an unreasonable annotation group, e.g., an artificially fabricated and false annotation group. A label vector of the negative sample is (0, 0, 0, . . . , 1, 0, 0). An element value corresponding to the Pattern_Outlier/Anomaly is 1 in the label vector. Other element values are 0.

During training, the training samples are input into an initial pattern determination model, and a loss function is constructed based on the output of the initial pattern determination model and the label. Parameters of the initial model are iteratively updated based on the loss function until the parameters of the initial pattern determination model satisfy a preset training condition, then the training is completed. A trained pattern determination model is obtained, and the trained pattern determination model is designated as the pattern determination model 410. The preset training condition may include, but is not limited to, the loss function converging, the training cycle reaching a threshold, or the like.

In 403, an intent verification result of the annotation group is determined based on the probability distribution.

In some embodiments, the reporting generation platform may recognize a statistical logic pattern with a highest probability in the probability distribution as a dominant pattern. Based on a rule set built into the reporting generation platform, the reporting generation platform determines whether the target annotation is compatible with the dominant pattern. In response to a determination that the target annotation is compatible with the dominant pattern, the reporting generation platform determines that the intent verification result is a reasonable intent. In response to a determination that the target annotation is incompatible with the dominant pattern, or the dominant pattern is Pattern_Outlier (i.e., the pattern determination model 410 considers the annotation group “disorganized”), the reporting generation platform determines that the intent verification result is an abnormal intent.

The rule set refers to a collection of logical rules used to determine whether the target annotation is compatible with the dominant pattern. For example, for the Pattern_Efficacy_Comparison pattern, a compatible annotation needs to have the following characteristics: an analyzed variable is the same as or similar to other annotations in the group, variables in filter conditions are consistent with other annotations in the group, a statistic (STAT) is common in efficacy analysis (e.g., a mean, a P value, and a confidence interval (CI)), or the like.

In 404: a prompt of the target annotation of the annotation group is generated based on the intent verification result.

In some embodiments, the reporting generation platform may generate the prompt of the target annotation of the annotation group based on the intent verification result. For example, the reporting generation platform remains silent for the reasonable intent; generates a corresponding warning for the abnormal intent.

In the present embodiment, by constructing the plurality of annotation groups and recognizing their statistical logic patterns, a deep understanding and recognition of the overall statistical intent of the annotations is implemented. Intelligent verification of logical consistency at the semantic level of the annotation data is realized. The depth and breadth of verification are improved. Furthermore, by calculating the probability distribution of the statistical logic pattern of the plurality of annotation groups and performing compatibility judgment, logic confusion and conclusion errors of the report caused by intent inconsistency are fundamentally avoided.

Some embodiments of the present disclosure, through an integrated, role-based, and automated reporting generation platform, transform the generation of clinical data reports from a mode highly dependent on individual expertise and manual operations into a streamlined, standardized, collaborative, and traceable production mode. This transformation yields fundamental improvements in efficiency, quality, compliance, and collaboration.

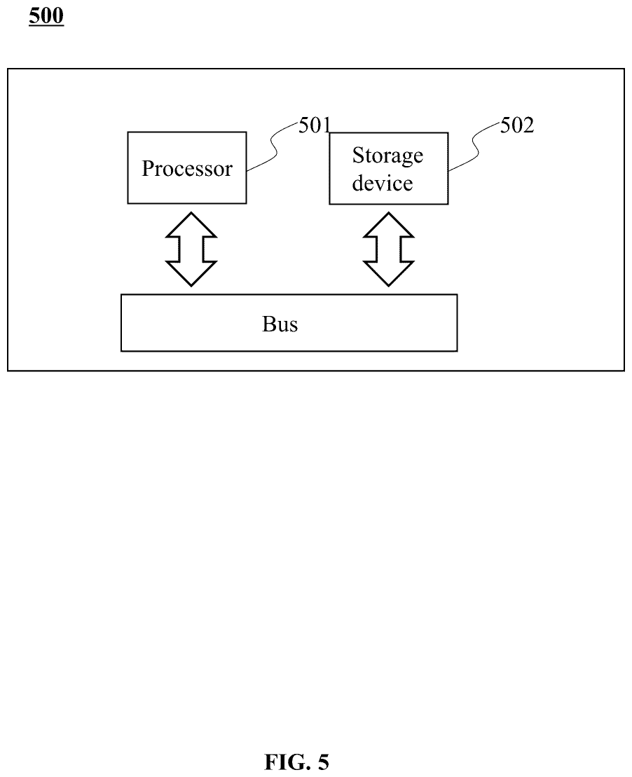

FIG. 5 is a schematic diagram illustrating an internal structure of a device for generating a report from clinical data according to some embodiments of the present disclosure. As shown in FIG. 5, the device 500 includes:

At least one processor 501.

And a storage device 502 communicatively connected to the at least one processor 501.

The storage device 502 stores instructions executed by the at least one processor 501. The instructions are executed by the at least one processor 501 to enable the at least one processor 501 to execute a method for generating a report from clinical data.

A non-transitory computer storage medium for generating a report from clinical data corresponding to FIG. 1 and provided in some embodiments of the present disclosure stores computer-executable instructions, when the computer instructions stored in the storage medium are read and executed by a computer, it causes the computer to implement a method for generating a report from clinical data.

Various embodiments in the present disclosure are described in a progressive manner. Identical or similar parts between the various embodiments may be referred to as each other. Each embodiment focuses on describing differences from other embodiments. In particular, for the Internet of Things device and media embodiments, since they are basically similar to the method embodiments, the description is relatively simple. For relevant parts, reference may be made to the description of the method embodiments.

The systems and media provided by the embodiments of the present disclosure have a one-to-one correspondence with the methods. Therefore, the systems and media also have beneficial technical effects similar to their corresponding methods. Since the beneficial technical effects of the methods have been described in detail above, the beneficial technical effects of the systems and media are not repeated here.

Those skilled in the art should understand that the embodiments of the present disclosure may be provided as a method, a system, or a computer program product. Therefore, the present disclosure may take the form of an entirely hardware embodiment, an entirely software embodiment, or an embodiment combining software and hardware aspects. Furthermore, the present disclosure may take the form of a computer program product implemented on one or more computer-usable storage media (including, but not limited to, disk storage devices, Compact Disc-Read-Only Memory (CD-ROM), optical storage devices, etc.) containing computer-usable program code.

Furthermore, the present disclosure may take the form of a computer program product implemented on one or more computer-usable storage media (including, but not limited to, the disk storage devices, the CD-ROM, the optical storage devices, etc.) containing computer-usable program code.

The present disclosure is described with reference to flowcharts and/or block diagrams of methods, devices (systems), and computer program products according to embodiments of the present disclosure. It should be understood that each process and/or block in the flowchart and/or block diagram, and combinations of processes and/or blocks in the flowchart and/or block diagram, can be implemented by computer program instructions. These computer program instructions can be provided to a processor of a general-purpose computer, a special-purpose computer, an embedded processor, or another programmable data processing device to produce a machine. Instructions executed by the processor of the computer or the other programmable data processing device produce means for implementing functions specified in one or more processes in the flowchart and/or one or more blocks in the block diagram.

These computer program instructions can also be stored in a computer-readable storage device that can direct the computer or the other programmable data processing device to function in a specific manner. These instructions stored in the computer-readable storage device produce an article of manufacture including instruction devices. The instruction devices implement functions specified in one or more processes in the flowchart and/or one or more blocks in the block diagram.

These computer program instructions can also be loaded into the computer or the other programmable data processing device. A series of operational steps is executed on the computer or the other programmable device to produce computer-implemented processing. These instructions executed on the computer or the other programmable device provide steps for implementing functions specified in one or more processes in the flowchart and/or one or more blocks in the block diagram.

In a typical configuration, a computing device includes one or more processors (e.g., Central Processing Units (CPUs)), input/output interfaces, network interfaces, and memories.

The memory may include forms of non-persistent memory in a computer-readable medium, Random Access Memory (RAM), and/or non-volatile memory, such as Read-Only Memory (ROM) or flash RAM. The memory is an example of a computer-readable medium.

The computer-readable medium includes permanent and non-permanent, removable and non-removable media and may be implemented using any manner or technology for information storage. The information can be computer-readable instructions, data structures, program modules, or other data. Examples of computer storage media include, but are not limited to, Phase-change Random Access Memory (PRAM), Static Random Access Memory (SRAM), Dynamic Random Access Memory (DRAM), other types of RAM, ROM, Electrically Erasable Programmable Read-Only Memory (EEPROM), flash memory or other memory technology, CD-ROM, Digital Versatile Disc (DVD) or other optical storage, magnetic cassettes, magnetic tape, magnetic disk storage or other magnetic storage devices, or any other non-transitory medium that can be used to store information accessible to the computing device. As defined herein, computer-readable media do not include transitory media, such as modulated data signals and carrier waves.

It should also be noted that the term “include,” “comprise,” or any variation thereof is intended to cover a non-exclusive inclusion. A process, method, article, or device that includes a list of elements includes those elements. It also includes other elements not explicitly listed or inherent to such process, method, article, or device. Without more constraints, an element defined by the phrase “include a/an . . . ” does not preclude the existence of additional identical elements in the process, method, article, or device that includes the element.

The foregoing descriptions are merely embodiments of the present disclosure, they are not intended to limit the present disclosure. Various modifications and variations to the present disclosure can be made by those skilled in the art. Any modification, equivalent replacement, or improvement made within the spirit and principle of the present disclosure shall be included within the scope of the claims of the present disclosure.

What is claimed is:

1. A method for generating a report from clinical data, comprising:
via a reporting generation platform, constructing a reporting generation project corresponding to clinical data to be processed and assigning corresponding roles and permissions to processing personnel; wherein the processing personnel include statistical analysts, reviewers, and annotation specialists;
constructing, by the statistical analysts, a template pending review based on a clinical trial design and publishing the template pending review;
performing, by the reviewers, online review on the template pending review and add online collaborative annotation, enabling the statistical analysts to obtain a template pending application by updating the template pending review based on the online collaborative annotation;
performing, by the annotation specialists, template annotation on the template pending application, enabling the reporting generation platform to automatically generate a corresponding SAS program based on the template annotation; and
performing the SAS program to obtain a report pending application.

2. The method of claim 1, wherein the constructing, by the statistical analysts, a template pending review based on a clinical trial design includes:
determining a keyword pending search based on the clinical trial design, and reading a report template library based on the keyword pending search to determine whether a relevant template whose matching degree against the keyword pending search exceeds a preset threshold exists with the report template library;
in response to the existence of the relevant template whose matching degree against the keyword pending search exceeds the preset threshold within the report template library, adjusting the relevant template based on the clinical trial design to obtain the template pending review; and
in response to the existence of the relevant template whose matching degree against the keyword pending search is less than the preset threshold within the report template library, generating a shell template and constructing the template pending review based on the clinical trial design.

3. The method of claim 1, wherein the method further includes:
labelling different character fields in a plurality of similar templates pending review and a plurality of similar templates pending application;
determining any one of the plurality of similar templates pending review or the plurality of similar templates pending application as an operational template to be processed, and designating all other templates as synchronized templates; and
during modification of the operational template to be processed, triggering an update component of the synchronized templates corresponding to the operational template to be processed to complete updates for the corresponding synchronized templates.

4. The method of claim 1, wherein the performing, by the annotation specialists, template annotation on the template pending application includes:
determining a parameter field to be annotated in the template pending application, and performing programmatic or natural language annotation on the parameter field to be annotated to generate a target annotation;
in response to determining that a logic verification function is triggered, performing logic verification on the target annotation to determine whether the target annotation complies with a writing standard; and
in response to determining that the target annotation complies with the writing standard, completing the template annotation on the template pending application.

5. The method of claim 4, wherein
the logic verification includes grammar logic verification and statistical logic verification, and the statistical logic verification includes:
determining annotation data to be processed corresponding to the target annotation and a SAS calculation macro corresponding to the target annotation by analyzing the target annotation;
retrieving a feature profile corresponding to the annotation data to be processed from a feature profile library built into the reporting generation platform based on the annotation data to be processed;
obtaining a result of the statistical logic verification based on the SAS calculation macro and the feature profile; and
generating a prompt of the target annotation based on the result of the statistical logic verification.

6. The method of claim 1, wherein the reporting generation platform automatically generating a corresponding SAS program based on the template annotation includes:
analyzing, via the reporting generation platform, the template annotation based on cross-platform computer programming language and a SAS parsing engine to convert the template annotation into a plurality of SAS calculation macros and calling parameters of the plurality of SAS calculation macros; and
summarizing the plurality of SAS calculation macros and the calling parameters of the plurality of SAS calculation macros to obtain the corresponding SAS program.

7. The method of claim 1, wherein after analyzing, via the reporting generation platform, the template annotation based on the cross-platform computer programming language and the SAS parsing engine, further includes:
analyzing the clinical data to be processed to parse the clinical data to be processed into variable units of corresponding filling parameters; and
summarizing the template annotation and the variable units to obtain a required dataset documentation.

8. The method of claim 6, wherein the analyzing, via the reporting generation platform, the template annotation based on cross-platform computer programming language and a SAS parsing engine to convert the template annotation into a plurality of SAS calculation macros and calling parameters of the plurality of SAS calculation macros includes:
parsing and recognizing annotated template to obtain structural information and annotation data of the annotated template; wherein the annotated template is the template pending application for which the annotation has been completed;
generating a structural file corresponding to the annotated template based on the structural information and the annotation data;
verifying the structural file based on a standard rule library built into the reporting generation platform to generate a verification result; and
in response to the verification result is passed, matching and determining, based on the structural file and via an analysis library built into the reporting generation platform, the plurality of SAS calculation macros and calling parameters of the plurality of SAS calculation macros.

9. The method of claim 6, wherein the summarizing the plurality of SAS calculation macros and the calling parameters of the plurality of SAS calculation macros to obtain the corresponding SAS program includes:
generating calling codes of at least one SAS calculation macro based on the plurality of SAS calculation macros and the calling parameters of the plurality of SAS calculation macros; and
generating the SAS program based on the calling codes of the at least one SAS calculation macro.

10. The method of claim 8, wherein the verifying the structural file based on a standard rule library built into the reporting generation platform to generate a verification result includes:
obtaining a plurality of target annotations and association annotations of the plurality of target annotations in the structural file, and constructing a plurality of annotation groups corresponding to the plurality of target annotations based on the plurality of target annotations and the association annotations of the plurality of target annotations;
for each of the plurality of annotation groups:
determining a probability distribution of a statistical logic pattern for the annotation group based on the annotation group;
determining an intent verification result of the annotation group based on the probability distribution; and
generating a prompt of a target annotation of the annotation group based on the intent verification result.

11. The method of claim 1, wherein after performing the SAS program to obtain the report pending application, the method further includes:
storing the report pending application and performing a SAS result dataset generated by the SAS program.

12. The method of claim 11, wherein the storing the report pending application and performing a SAS result dataset generated by the SAS program includes:
constructing independent Key variables for each of a plurality of result cells in the report pending application, respectively, wherein the Key variables store metadata information of the plurality of result cells, the metadata information includes a row/column coordinate, an input dataset name, a variable name, a variable value or label, and a generation condition;
the method further includes:
in response to version comparison instructions, matching the Key variables to the corresponding result cells of the report pending applications in different versions and displaying differences in the corresponding result cells.

13. The method of claim 1, wherein the method further includes:
generating and saving a communication log when the reviewers perform online review on the template pending review and the report pending application and add the online collaborative annotation.

14. A device for generating a report from clinical data, comprising:
at least one processor;
and, a storage device communicatively connected to the at least one processor;
wherein the storage device stores instructions executed by the at least one processor, and the at least one processor is configured to:
via a reporting generation platform, construct a reporting generation project corresponding to clinical data to be processed and assigning corresponding roles and permissions to processing personnel; wherein the processing personnel include statistical analysts, reviewers, and annotation specialists;
construct, by the statistical analysts, a template pending review based on a clinical trial design and publishing the template pending review;
perform, by the reviewers, online review on the templates pending review and add online collaborative annotation, enabling the statistical analysts to obtain a template pending application by updating the template pending review based on the online collaborative annotation;
perform, by the annotation specialists, template annotation on the template pending application, enabling the reporting generation platform to automatically generate a corresponding SAS program based on the template annotation; and
perform the SAS program to obtain a report pending application.

15. A non-transitory computer storage medium for generating a report from clinical data storing instructions, the instructions, when executed by at least one processor, causing the at least one processor to implement a method comprising:
via a reporting generation platform, constructing a reporting generation project corresponding to clinical data to be processed and assigning corresponding roles and permissions to processing personnel; wherein the processing personnel include statistical analysts, reviewers, and annotation specialists;
constructing, by the statistical analysts, a template pending review based on a clinical trial design and publishing the template pending review;
performing, by the reviewers, online review on the templates pending review and add online collaborative annotation, enabling the statistical analysts to obtain a template pending application by updating the template pending review based on the online collaborative annotation;
performing, by the annotation specialists, template annotation on the template pending application, enabling the reporting generation platform to automatically generate a corresponding SAS program based on the template annotation; and
performing the SAS program to obtain a report pending application.
